(19) United States
(12) Patent Application Publication
(10) Pub. No.: US 2006/0052303 A1
(43) Pub. Date: Mar. 9, 2006

(54) Use of thyrotropin for regeneration of bone

(76) Inventors: Kuber T. Sampath, Holliston, MA (US); John M. McPherson, Hopkinton, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/190,465

(22) Filed: Jul. 27, 2005

Related U.S. Application Data
(60) Provisional application No. 60/591,464, filed on Jul. 27, 2004.

(51) Int. Cl. A61K 38/22

(52) U.S. Cl. 514/12

(57) ABSTRACT

The invention provides methods for treating or preventing bone degenerative disorders. The disorders treated or prevented include, for example, osteopenia, osteomalacia, osteoporosis, osteomyeloma, osteodystrophy, Paget's disease, osteogenesis imprerfecta, and bone degenerative disorders associated with chronic renal disease, hyperparathyroidism, high levels of endogenous thyrotropin, and long-term use of corticosteroids. The disclosed therapeutic methods include administering to a mammal a TSHR agonist in an amount effective to: (1) treat or prevent a bone degenerative disorder; (2) slow bone deterioration; (3) restore lost bone; (4) stimulate new bone formation; and/or (5) maintain bone mass and/or bone quality. TSHR agonists such as thyrotropin and its modified forms are provided along with other compounds, such as anti-resorptive agent and bone metabolic agents.

This application claims the benefit of U.S. provisional patent application No. 60/591,464, filed Jul. 27, 2004, which is incorporated herein by reference.

### TECHNICAL FIELD

The technical field of the invention relates to the therapeutic uses of thyroid stimulating hormone (TSH; thyrotropin) in the treatment of bone degenerative disorders such as osteoporosis, osteopenia, osteomalacia, and osteodystrophy.

### BACKGROUND OF THE INVENTION

Thyroid stimulating hormone (TSH; thyrotropin) is an endocrine hormone secreted by the anterior pituitary gland in response to a signal from the hypothalamus. Thyrotropin is responsible for thyroid follicle development and thyroid hormone production. It binds to the G-protein coupled receptor, TSHR, on epithelial cells in the thyroid gland, thereby stimulating the gland to synthesize and release thyroid hormones. TSHR is expressed in several tissues other than the thyroid gland including bone marrow cells, lymphocytes, thymus, testes, kidney, brain, and adipose, lymphoid, and skeletal tissues. Production of thyrotropin is controlled by a classical negative feedback loop mechanism, in which high blood levels of thyroid hormones inhibit thyrotropin secretion.

A recombinantly produced human thyrotropin, Thyrogen® (Genzyme Corp.), has been used in thyroid scanning and thyroglobulin level testing in the follow-up of patients with well-differentiated thyroid cancer. Additional proposed clinical uses include thyrotropin stimulation tests (e.g., testing thyroid reserve) and the treatment of nonthyroidal illness syndrome, thyroid cancer, and large euthyroid goiter by thyrotropin-stimulated radioiodine ablation.

The relationship between thyroid hormones and bone was first recognized in the 1890's, when it was first observed that hyperthyroidism is associated with a higher rate of bone fractures (Bauer et al. (2001) Ann. Inter. Med., 134:561-568).

Throughout adult life, bone continually undergoes a turnover through the coupled processes of bone formation and resorption. Bone resorption is mediated by bone resorbing cells, osteoclasts, which are formed by mononuclear phagocytic precursor cells. New bone replacing the lost bone is deposited by bone-forming cells, osteoblasts which are formed by mesenchymal stromal cells. Various other cell types that participate in the remodeling process are tightly controlled by systemic factors (e.g., hormones, lymphokines, growth factors, vitamins) and local factors (e.g., cytokines, adhesion molecules, lymphokines, and growth factors). The proper spatiotemporal coordination of the bone remodeling process is essential to the maintenance of bone mass and integrity. A number of bone degenerative disorders are linked to an imbalance in the bone remodeling cycle which results in abnormal loss of bone mass (osteopenia) including metabolic bone diseases, such as osteoporosis, osteoplasia (osteomalacia), osteodystrophy, Paget's disease, chronic renal disease, and primary and secondary hyperparathyroidism.

Thyroid disease is one of the most common endocrine problems. Exogenous administration of a thyroid hormone, L-thyroxine, to suppress thyrotropin is a therapy widely used to inhibit progression or recurrence of papillary or follicular thyroid cancer and other hyperthyroid conditions. The effects on bone in hyperthyroid dysfunctions have been attributed to the levels of thyroid hormones, which are directly implicated in the regulation of calcium homeostasis. Hyperthyroid patients exhibit low (or undetectable) circulating levels of thyrotropin which are associated with loss of bone. Additionally, thyroxin is known to induce osteoporosis in some patients. Hypothyroidism, on the other hand, is associated with high bone mass and elevated levels of thyrotropin.

The exact role of thyrotropin in bone homeostasis has not been elucidated. Mice genetically deficient in thyroid hormones or α1/β thyroid hormone receptor (TR) have normal remodeling phenotype despite abnormalities in skeletal morphogenesis and growth of plate, Gother et al. (1999) Genes and Devel., 13:1329-1341. Further, TSHR-deficient hetero- and homozygous mice exhibit high turnover bone remodeling which results in reduced bone mass and focal bone sclerosis (Abe et al. (2003) Cell, 115:151-162). Based on in vitro studies with bone cells derived from TSHR-deficient mice, thyrotropin has been suggested to have a direct negative regulatory effect on both the anabolic and the catabolic arms of the bone remodeling process. Specifically, thyrotropin has been reported to suppress both osteoclast formation and osteoblast differentiation (Abe, supra).

Conventional therapies for the treatment of bone degenerative disorders include calcium supplements, estrogen, calcitonin, and bisphosphonates. Vitamin D3 and its metabolites, known to enhance calcium and phosphate absorption, also are being tried. However, none of these therapies stimulate formation of new bone tissue. Moreover, these agents have only a transient effect on bone remodeling. Thus, while in some cases the progression of the disease may be halted or slowed, patients with significant bone deterioration remain at risk. This is particularly prevalent in disorders such as post-menopausal osteoporosis where at diagnosis structural deterioration of the bone often has already started to occur.

Therefore, there exists a need to develop new therapeutic methods for treating and preventing bone disorders.

### SUMMARY OF THE INVENTION

The invention is based, in part, on the discovery and demonstration that systemic administration of thyrotropin to ovariectomized rats immediately following surgery is effective in slowing the loss of bone that occurs following estrogen deficiency. Ovariectomy-induced osteopenia is a well-validated model of early post-menopausal osteopenia. Therefore, the present disclosure demonstrates for the first time that thyrotropin has a therapeutic effect on bone.

Accordingly, the invention provides methods for treating or preventing bone degenerative disorders. The disorders treated or prevented include, for example, osteopenia, osteomalacia, osteoporosis, osteomyeloma, osteodystrophy, Paget's disease, osteogenesis imperfecta, bone sclerosis, aplastic bone disorder, humoral hypercalcemic myeloma, multiple myeloma and bone thinning following metastasis. The disorders treated or prevented further include bone degenerative disorders associated with hypercalcemia, chronic renal disease (including end-stage renal disease), kidney dialysis, primary or secondary hyperparathyroidism, and long-term use of corticosteroids.

The disclosed methods include administering to a mammal a TSHR agonist in an amount effective to:

(1) treat or prevent a bone degenerative disorder;

(2) slow bone deterioration;

(3) restore lost bone;

(4) stimulate new bone formation; and/or;

(5) maintain bone (bone mass and/or bone quality).

In certain embodiments, the TSHR agonist is thyrotropin or a biologically active analog thereof. In illustrative embodiments, thyrotropin is recombinantly produced human thyrotropin, e.g., thyrotropin alfa. The invention further provides assays for evaluating efficacy of a TSHR agonist for treatment of a bone degenerative disorder. Methods of administration, compositions, and devices used in the methods of the inventions are also provided.

The invention will be set forth in the following description, and will be understood from the description, or may be learned by practice of the invention.

### BRIEF DESCRIPTION OF THE FIGURES

FIGS. 1A and 1B show amino acid sequences of human thyrotropin α (FIG. 1A) and β (FIG. 1B) subunits. Three known N-linked glycosylation sites are indicated with asterisks. Cysteines forming disulfide bridges of the cysteine knot structure are highlighted in black. The proteolytic cleavage site producing cleavage of the C-terminal 6 amino acids in the β subunit resulting in 112-amino acid product is marked with an arrow. β-hairpin loops are underlined by a single line; α-helix is underlined with a double line; the βC88-βC105 “seatbelt” structure is underlined by a dashed line.

FIG. 2 illustrates the tertiary structure of thyrotropin showing several domains and point mutations that have been implicated in biological activity. (The figure is as shown in Leitolf et al. (2000) J. Biol. Chem., 275:27457-65.) The α subunit backbone is shown as a gray line, and the β subunit chain is shown as a black line. The functionally important domains are marked as follows: the peripheral β-hairpin loops are marked as αL1, αL3 in the α subunit; βL1, βL3 in the β subunit; two long loops are αL2 with α-helical structure and βL2. Circles represent positions of amino acid residues (α13, α14, α16, and α20 in αL1; α64, α66, α73, and α81 in αL3; β58, β63, and β69 in βL3), substitution of which in human thyrotropin with basic residues results in enhancement of biological activity. β-hairpin loops are underlined by a single line; α-helix is underlined with a double line; the βC88-⊕C105 “seatbelt” structure is underlined by a dashed line.

FIGS. 3A, 3B, and 3C illustrate alternate structures of N-linked carbohydrates on thyrotropin. Blackened diamonds represent N-acetylglucosamine; blackened circles represent mannose; blackened squares represent fucose; hatched circles represent N-acetylgalactosamine; hatched diamonds represent galactose; SO₄ denotes a sulfated sugar; NeuAc denotes a sialated sugar. FIG. 3A depicts a typical oligosaccharide structure of bovine thyrotropin. FIG. 3B depicts a typical oligosaccharide structure of pituitary gland derived human thyrotropin. FIG. 3C depicts a typical oligosaccharide structure of recombinant thyrotropin expressed in Chinese hamster ovary cells.

FIGS. 4A and 4B show alignments of amino acid sequences from several species for thyrotropin α (FIG. 4A) and β (FIG. 4B) subunits.

FIG. 5 provides a number of aligned amino acid sequences derived from various species. The aligned regions correspond to amino acids 10-28 of human thyrotropin α subunit.

FIG. 6 shows results of a bone mineral density (BMD) analysis of total body in live animals. Rats were ovariectomized (OVX) and treated with 0.7, 7, or 70 μg per rat Thyrogen® or with estrogen for up to 8 weeks following surgery. BMD analysis was performed every 2 weeks. Asterisks denote a statistically significant difference as compared to the OVX group.

FIG. 7 shows results of a BMD analysis of hind limbs. Animals were treated as described for FIG. 6.

FIG. 8 shows results of a BMD analysis of the lumber spine. Animals were treated as described for FIG. 6.

FIG. 9 shows results of a BMD analysis of the proximal region of femur, performed ex vivo. Animals were treated as described for FIG. 6.

FIG. 10 shows results of a BMD analysis of the distal region of femur, performed ex vivo. Animals were treated as described for FIG. 6.

FIG. 11 shows results of a BMD analysis of the total tibia, performed ex vivo. Animals were treated as described for FIG. 6.

FIG. 12 shows results of a BMD analysis of the lumbar spine, performed ex vivo. Animals were treated as described in FIG. 6.

FIG. 13 shows results of an in vivo dual-energy X-ray absorptiometry (DEXA) analysis of total body BMD from bone restoration study. Rats were ovariectomized and treated with 0.01; 0.1; or 0.3 μg of rat TSH (as indicated) starting seven months after surgery and continuing for 16 weeks.

FIG. 14 shows results of an in vivo DEXA analysis of hind limbs BMD. Animals were treated as described for FIG. 13.

FIG. 15 shows results of an ex vivo DEXA analysis of proximal femur BMD. Animals were treated as described for FIG. 13.

FIG. 16 shows results of an ex vivo DEXA analysis of distal tibia BMD. Animals were treated as described for FIG. 13.

FIG. 17 shows results of an ex vivo lumbar spine BMD analysis. Animals were treated as descrbied for FIG. 13.

FIG. 18 shows results of a microCT analysis of bone volume/trabecular volume (BV/TV) analysis. Animals were treated as described for FIG. 13.

FIG. 19 shows results of a microCT analysis of trabecular thickness. Animals were treated as described for FIG. 13.

FIG. 20 shows results of a microCT analysis of trabecular number. Animals were treated as described for FIG. 13.

FIG. 21 shows results of a microCT analysis of cortical thickness. Animals were treated as described for FIG. 13.

### BRIEF DESCRIPTION OF THE SEQUENCES

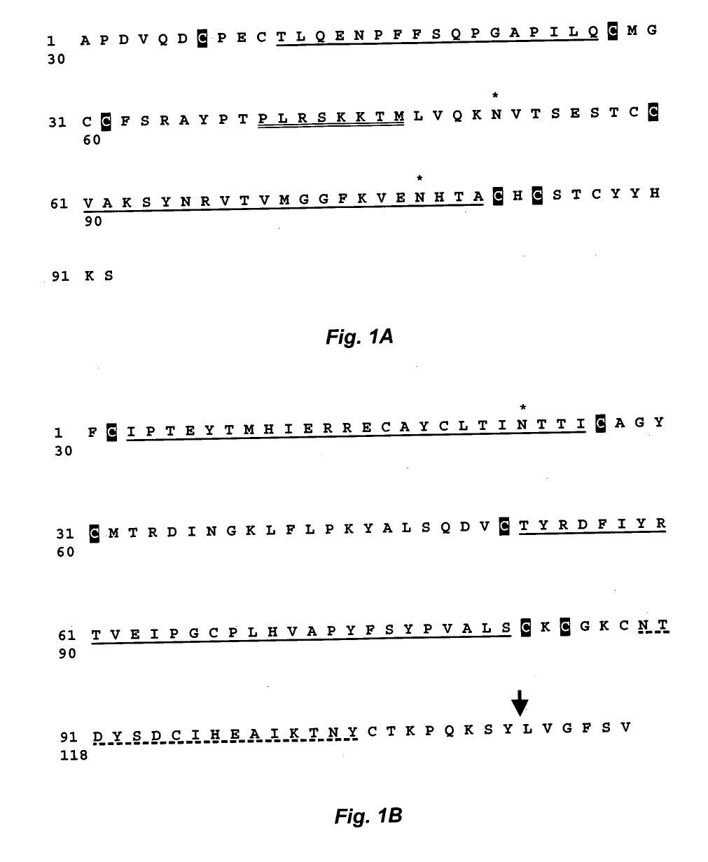

SEQ ID NO:1 is an amino acid sequence of the α subunit of human thyrotropin as depicted in FIG. 1A.

SEQ ID NO:2 is a nucleotide sequence encoding the α subunit of human thyrotropin precursor. Nucleotide residues 73 to 351 encode SEQ ID NO:1.

SEQ ID NO:3 is an amino acid sequence of the β subunit of human thyrotropin as depicted in FIG. 1B.

SEQ ID NO:4 is a nucleotide sequence encoding the β subunit of human thyrotropin precursor. Nucleotide residues 61 to 417 encode SEQ ID NO:3.

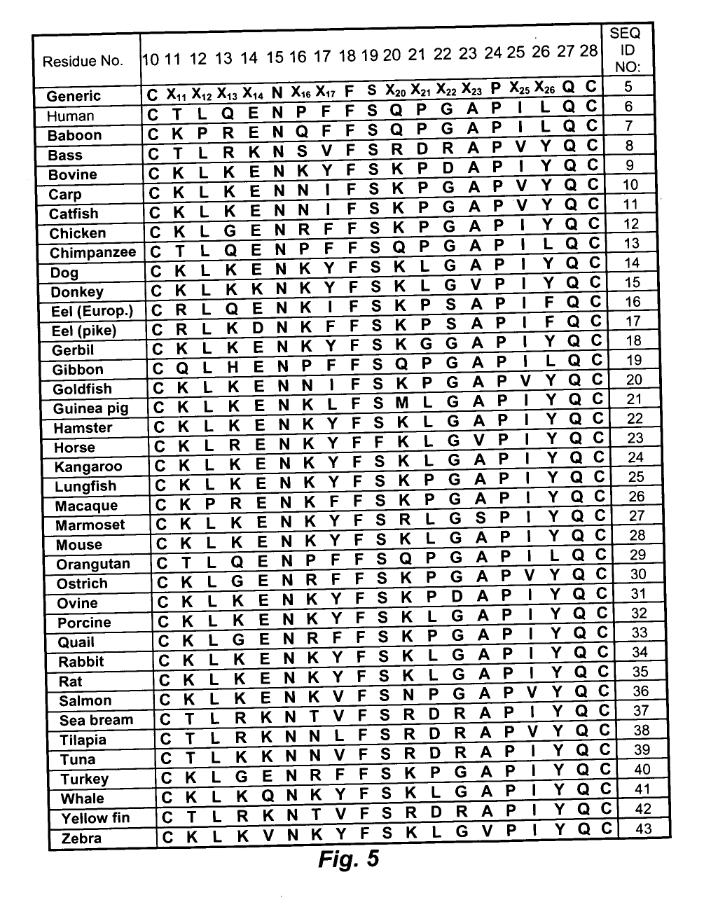

SEQ ID NO:5 is a genericized amino acid sequence in the L1 loop of the α subunit, corresponding to amino acids 10-28 of human thyrotropin (see FIG. 5).

SEQ ID NOs:6-43 are amino acid sequences derived from various species (see FIG. 5) in the L1 loop of the α subunit, corresponding to amino acids 10-28 of human thyrotropin.

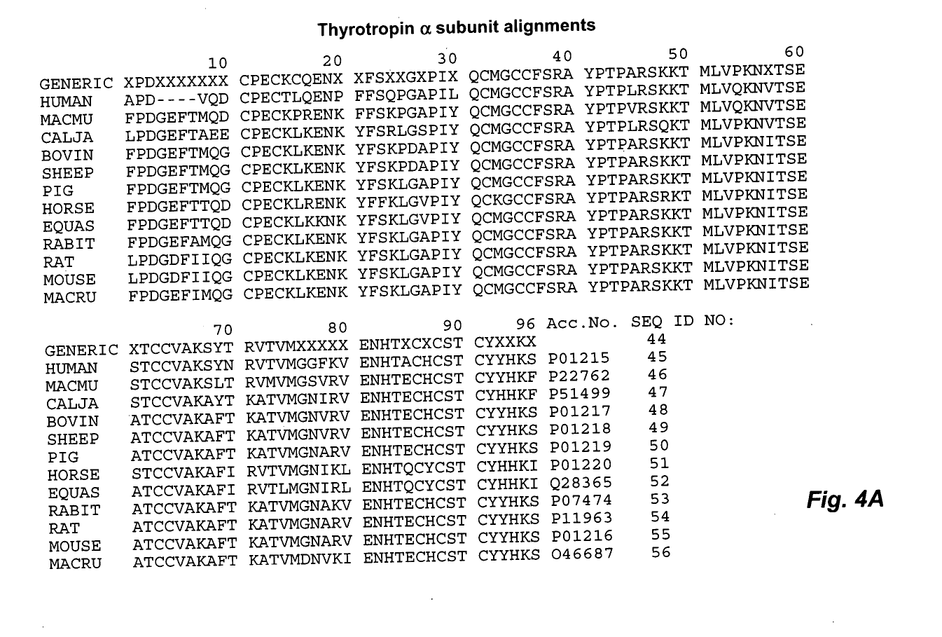

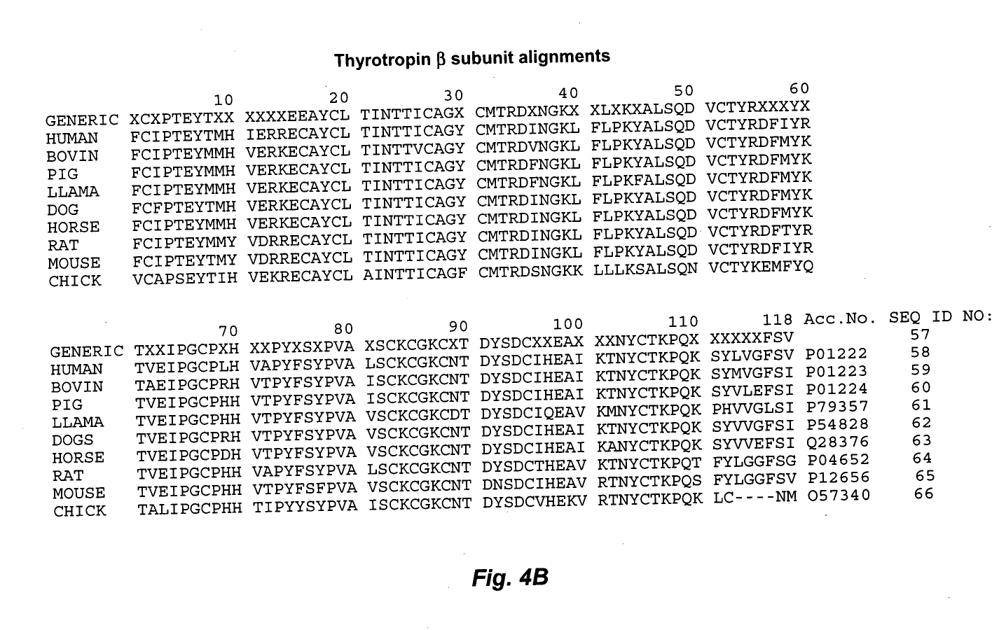

SEQ ID NO:44 is a generic sequence of the full length thyrotropin α subunit based on the alignment shown in FIG. 4A.

SEQ ID NOs:45-56 are amino acid sequences of the α subunit thyrotropin derived from various species.

SEQ ID NO:57 is a generic sequence of the full length thyrotropin β subunit based on the alignment shown in FIG. 4B.

SEQ ID NOs:58-66 are amino acid sequences of the β subunit thyrotropin derived from various species.

### DETAILED DESCRIPTION OF THE INVENTION

Compositions used in the methods of the invention include TSHR agonists.

The term “TSHR agonist” refers to a compound or composition (regardless of source or mode of production) that enhances thyrotropin signaling pathway. TSHR agonists may stimulate the TSHR-mediated signaling by themselves, and/or stimulate TSHR-mediated signaling by enhancing the biological activity of endogenous thyrotropin or another administered (i.e., exogenous) TSHR agonist. TSHR agonists may also activate or inactivate genes that are specific to thyrotropin down stream signaling. Certain TSHR agonists specifically bind the thyrotropin receptor which then transduces TSHR-mediated intracellular signaling in thyrotrophs or other cells naturally expressing TSHR or cells modified to express TSHR. The term “specific binding” and its cognates refer to an interaction with an affinity constant Ka of at least, for example, 10⁵, 0.5×10⁶, 10⁶, 0.5×10⁷, 10⁷, 0.5×10⁷, 0.5×10⁸, 10⁸, 0.5×10⁹, 10⁹ M⁻¹ or higher as determined under appropriate conditions (e.g., as described in the Examples).

TSHR agonists include, for example, thyrotropin and thyrotropin analogs, anti-TSHR antibodies, and small molecules as will be described below. Thyrotropin analogs include proteinaceous thyrotropin analogs such as modified thyrotropin and non-naturally occurring biologically active fragments of thyrotropin and of modified thyrotropin.

Assays for determining the biological activity of a TSHR agonist are known in the art. For example, the biological activity may be determined in a cell-based assay as described in the Examples. In such an assay, TSHR-mediated signaling activity is determined based on the level of intracellular 3′,5′-cyclic adenosine monophosphate (cAMP). The effect of a test agent on the level of cAMP is measured in cells expressing a functional TSHR and, and optionally, a cAMP-responsive reporter gene construct. Expression of a functional TSHR has been previously accomplished as described, for example, in Akamizu et al. (1990) Proc. Natl. Acad. Sci. USA., 87:5677-5681; Frazier et al. (1990) Mol. Endocrinol., 4:1264-1276; Libert et al. (1989) Biochem. Biophys. Res. Commun., 165:1250-1255; Libert et al. (1990) Mol. Cell. Endocrinol., 68:R15-R17; Misrahi et al. (1990) Biochem. Biophys. Res. Commun., 166: 394-403; Nagayama et al. (1989) Biochem. Biophys. Res. Commun., 165: 1184-1190; Parmentier et al. (1989) Science, 246: 1620-1622; Perret et al. (1990) Biochem. Biophys. Res. Commun., 28:171(3):1044-50; and in U.S. Pat. No. 6,361,992 (see, e.g., assays employing CHO cells and CHO-J09 clone, in particular).

The biological activity of thyrotropin alfa is determined by a cell-based assay. In this assay, cells expressing a functional thyrotropin receptor and a cAMP-responsive element coupled to a heterologous reporter gene, such as, for example, luciferase, are utilized. The measurement of the reported gene expression provides an indication of thyrotropin activity. The specific activity of thyrotropin alfa is determined relative to a reference material that is calibrated against the World Health Organization (WHO) human pituitary derived thyrotropin reference standard NIBSC 84/703 using an in vitro assay that measures the amount of cAMP produced by a bovine thyroid microsome preparation in response to thyrotropin alfa. The specific activity of thyrotropin alfa is typically in the range of 4-12 lU/mg as determined by a cell-based assay.

TSHR agonists include thyrotropin and thyrotropin analogs.

Thyrotropin, used in the methods of the invention, is purified naturally occurring thyrotropin or recombinantly or synthetically produced thyrotropin. In illustrative embodiments, thyrotropin is “thyrotropin alfa” (marketed as Thyrogen®).

Thyrotropin is composed of two non-covalently bound subunits, α and β. Free α and β subunits have essentially no biological activity. The α subunit is also present in two other pituitary glycoprotein hormones, follicle-stimulating hormone and luteinizing hormone, and in primates, in the placental hormone chorionic gonadotropin. The unique β subunit confers receptor specificity to the dimer. The sequences of the thyrotropin α and β subunits are highly conserved from fish to mammals. For example, human and bovine thyrotropins share 70% homology in the β subunit, and 89% in the α subunit.

Amino acid sequences of human thyrotropin α (SEQ ID NO:1) and β (SEQ ID NO:3) subunits are shown in FIGS. 1A and 1B, respectively. Their respective encoding nucleic acids are provided as SEQ ID NO:2 (nucleotide residues 73 to 351 encode SEQ ID NO:1) and SEQ ID NO:4 (nucleotide residues 61 to 417 encode SEQ ID NO:3). (The additional nucleotide sequences at the 5′ end encode signal peptides.)

Cysteines forming disulfide bridges of the cysteine knot structure are highlighted in black in FIGS. 1A and 1B. The cysteine knot motif is formed by Cys34-Cys88, Cys9-Cys57, Cys38-Cys90 and Cys23-Cys72, Cys94-Cys100, Cys26-Cys100. The cysteine knot has been recognized as important for intracellular stability but not essential for receptor binding or biological activity.

The database accession numbers of full-length α subunit sequences from various species and references to sequences in the Sequence Listing are as follows: human (P01215; SEQ ID NO:45); rhesus macaque (P22762; SEQ ID NO:46); marmoset (P51499; SEQ ID NO:47); bovine (P01217; SEQ ID NO:48); sheep (P01218; SEQ ID NO:49); pig (P01219; SEQ ID NO:50); horse (P01220; SEQ ID NO:51); donkey (Q28365; SEQ ID NO:52); rabbit (P07474; SEQ ID NO:53); rat (P11963; SEQ ID NO:54); mouse (P01216; SEQ ID NO:55); kangaroo (O46687; SEQ ID NO:56). Accordingly, in certain embodiments, thyrotropin comprises any of the SEQ ID NOs:45-56.

The database accession numbers of full-length β subunit sequences from various species and references to sequences in the Sequence Listing are as follows: human (P01222; SEQ ID NO:58); bovine (P01223; SEQ ID NO:59); pig (P01224; SEQ ID NO:60); llama (P79357; SEQ ID NO:61); dog (P54828; SEQ ID NO:62); horse (Q28376; SEQ ID NO:63); rat (P04652; SEQ ID NO:64); mouse (P12656; SEQ ID NO:65); chicken (O57340; SEQ ID NO:66); hamster (Q62590); and fish (P37240; O73824; Q08127). Accordingly, in certain embodiments, thyrotropin comprises any of the SEQ ID NOs:57-66 and Q62590, P37240, O73824, and Q08127.

In some embodiments, thyrotropin is recombinantly produced. Thyrogen® (“thyrotropin alfa” for injection) contains a highly purified recombinant form of human thyrotropin, a glycoprotein which is produced by recombinant DNA technology.

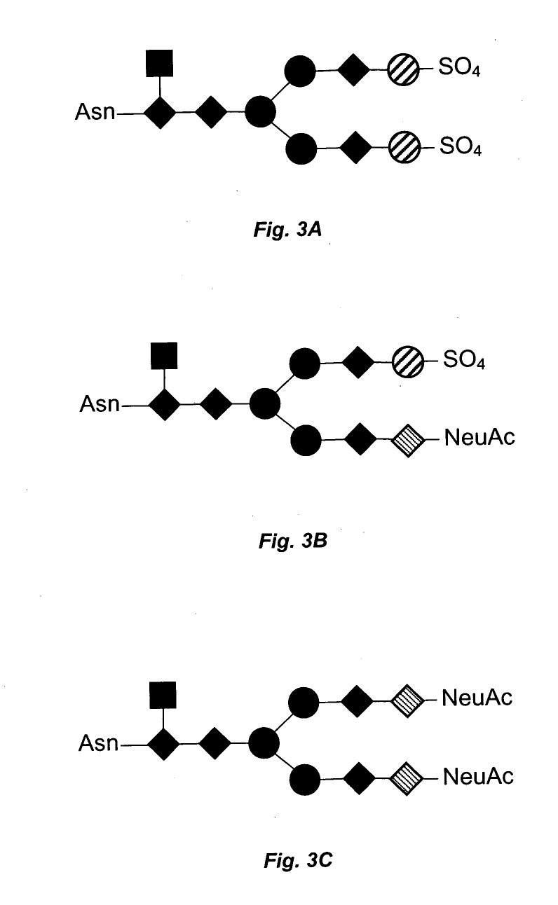

Both thyrotropin alfa and naturally occurring human pituitary thyroid stimulating hormone are synthesized as a mixture of glycosylation variants. FIGS. 3A-3C illustrate alternate structures of N-linked carbohydrates on thyrotropin. Recombinant thyrotropin produced in CHO cells is sialated not sulfated because these cells lack GalNAc- and sulfo-transferases. Although the glycosylation of recombinantly produced thyrotropin is not identical to naturally occurring thyrotropin, its biological activity is similar to that of pituitary thyrotropin. Accordingly, thyrotropin and its analogs of the invention may comprise a heterogeneous mix of oligosaccharide structures.

TSHR agonists useful in the methods of the invention include proteinaceous thyrotropin analogs. Proteinaceous thyrotropin analogs include modified thyrotropin and non-naturally occurring biologically active fragments of thyrotropin and of modified thyrotropin. Illustrative procedures for screening proteinaceous thyrotropin analogs are described in the Examples. Modified thyrotropin includes non-naturally occurring variants of thyrotropin in which (1) at least one but fewer than 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids are substituted or deleted in the α subunit and/or (2) at least one but fewer than 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids have been substituted or deleted in the β subunit; as compared to naturally occurring thyrotropin. For example, one or more amino acids may be substituted in human thyrotropin by a corresponding residue from another species. The term “corresponding” or its cognates, when used in reference to a position of an amino acid in a first amino acid sequence relative to a second amino acid sequence, refers to the amino acid residue in the second sequence that aligns with that position in the first sequence when both sequences are optimally aligned (i.e., the maximal possible number of amino acids in both sequences match). Unless otherwise stated, all amino acid positions refer to human sequences and corresponding amino acids in other species and modified forms of thyrotropin.

FIG. 5 shows alignments of amino acid sequences from several species for thyrotropin α (FIG. 4A) and β (FIG. 4B) subunits. The variable amino acids can but do not need to be derived from corresponding amino acids from other species, for example, as shown in FIGS. 4A, 4B, and 5.

Accordingly, in some embodiments a thyrotropin analog comprises SEQ ID NO:44 and/or SEQ ID NO:57.

FIG. 5 contains a number of aligned amino acid sequences derived from the α subunit, which correspond to amino acids 10-28 of human sequence. This region in the αL1 has been recognized as one of the regions important for biological activity. Notably, bovine thyrotropin is significantly more active than its human version, presumably due to the amino acid differences in this region. Based on the alignment, modified thyrotropin comprises the amino acid sequence as set out in SEQ ID NO:6, wherein X11 is T, Q, K, R, or another amino acid; X12 is L, P, or another amino acid; X13 is Q, H, R, K, G, or another amino acid; X14 is E, V, K, Q, D, or another amino acid; X16 is P, Q, K, R, N, S, T, or another amino acid; X17 is F, Y, L, I, V, or another amino acid; X20 is Q, K, R, M, N, or another amino acid; X21 is P, L, G, D, or another amino acid; X22 is G, D, R, S, or another amino acid; X23 is A, S, V, or another amino acid; X25 is I, V, or another amino acid; X26 is L, Y, F, or another amino acid; all independently variable.

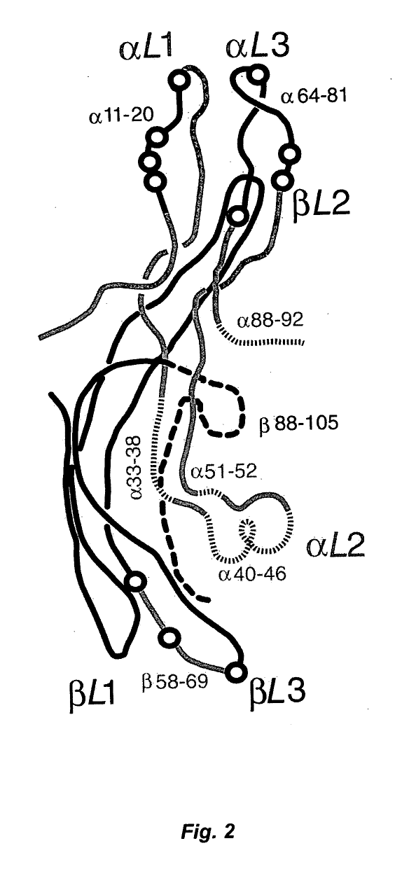

Further examples of modified thyrotropin include “TSH superagonists” described in U.S. Pat. No. 6,361,992. Such modified thyrotropin may contain mutations of one or more amino acid at residues 11, 13, 14, 16, 17, 20 L1 in the α subunit (αL1 region) and amino acid residues 13, 20, 58, 63, and 69 in the β subunit. Substitution of these residues in human thyrotropin with basic residues results in enhancement of biological activity (also known as “gain-of function” analogs). FIG. 2 illustrates the location of certain “gain-of-function” mutations on the tertiary structure human thyrotropin (i.e., amino acid residues α13, α14, α16, and α20 in αL1; α64, α66, α73, and α81 in αL3; β58, β63, and β69 in βL3). For example, human thyrotropin with four substitutions in the α subunit (Q13K, E14K, P16K, Q20K) and an additional substitution in the β subunit (L69R) shows 95 times greater biological activity as compared to the wild-type thyrotropin. The same four substitutions in the α subunit and three substitutions in the β subunit (I58K, E63, L69T) results a 100-fold increase of biological activity.

The following amino acids have been recognized as playing an important role in binding TSHR and/or the biological activity of thyrotropin. In the α subunit: α10-28; α33-38; α-helix (α40-46); αK51; αN52 and αN78 carrying the N-linked sugars; the C-terminus (α88-92). In the β subunit: βN23 carrying the N-linked sugars; Kautmann's loop” (β31-52) and particularly the “seat-belt” region (β88-105). (For review of structure-functional studies, see, e.g., Szkudlinski et al. (2002) Physiol. Rev., 82:473-502.)

Three known N-linked glycosylation sites are indicated with asterisks in FIGS. 1A and 1B. The N-linked glycosylation on the molecule determine the level of its biological activity. Deglycosylation of human chorionic gonadotropin and bovine thyrotropin results in increased receptor binding but decreased receptor signal transduction. Additionally, the proteolytic cleavage site producing cleavage of the C-terminal 6 amino acids in the β subunit resulting in a 112-amino acid product is marked with an arrow in FIG. 1B. This cleavage is found in the purified human pituitary thyrotropin and does not seem to affect the activity of the hormone.

Proteinaceous TSHR antagonist includes biologically active fragments of thyrotropin and its modified forms.

Accordingly, in some embodiments, a thyrotropin analog comprises:

(1) any one or any two of the three asparagines that correspond to amino acid residues αN52, αN78, and βN23, or alternatively, all three asparagines;

(2) one or more amino acid (aa) sequences selected from aa 10-28 of SEQ ID NO:1, aa 33-38 of SEQ ID NO:1, aa 40-46 of SEQ ID NO:1, aa 88-92 of SEQ ID NO:1, 31-52 of SEQ ID NO:2, 88-105 of SEQ ID NO:2 and amino acid sequences corresponding to SEQ ID NOs:45-56 and SEQ ID NO:58-66;

(3) any one of amino acid sequences SEQ ID NOs:6-43;

(4) SEQ ID NO:44;

(5) SEQ ID NO:57;

(6) SEQ ID NO:44 and SEQ ID NO:57;

(7) any one of amino acid sequences SEQ ID NOs:45-56;

(8) any one of amino acid sequences SEQ ID NOs:58-66;

(7) aa 1-112 of SEQ ID NO:3 or corresponding amino acids in any one of SEQ ID NOs:57-66; or

(8) any one of (1), (2), (3), (4), (5), (6), and (7) that comprises at least 20, 30, 40, 50, 60, 70, 80, 90 amino acids that correspond to either human thyrotropin α subunit or β subunit.

Proteinaceous thyrotropin analogs further include agonistic anti-TSHR antibodies. The term “antibody” refers to an immunoglobulin (Ig) that specifically binds to TSHR. The term also refers to a portion or a fragment of such an immunoglobulin so long as it retains specificity for TSHR. Antibodies useful in the present invention are not limited with regard to the source or method of production. Most typically, monoclonal antibodies are used. Most commonly, Ig type G (IgG) is used. Antibodies may be fully human; fully murine; CDR-grafted (e.g., humanized), chimeric (e.g., comprising human variable domain and murine constant domains), synthetic, recombinant, hybrid, or mutated. Producing antibodies is well within the ordinary skill of an artisan (see, e.g., Antibody Engineering, ed. Borrebaeck, 2nd ed., Oxford University Press, 1995). Examples of agonistic anti-TSHR antibodies include human monoclonal thyroid stimulating autoantibody (see, e.g., Sanders et al. (2003) Lancet, 362(9378):126-128 and Kin-Saijo et al. (2003) Eur. J. Immunol., 33:2531-2538) mouse monoclonal anti-TSHR antibody with stimulating activity (see, e.g., Costagliola et al. (2000) BBRC, 299(5): 891-896).

Methods of making thyrotropin analogs are known in the art. The analogs can be synthesized chemically or recombinantly. Recombinant thytropin can be produced recombinantly as described, for example, by Cole et al. (1993) Bio/Technology, 11:1014-1024. Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include CHO cells, HeLa cells, baby hamster kidney cells, NS0 mouse melanoma cells, and many others. For other cells suitable for producing TSHR agonists, see, e.g., Gene Expression Systems, eds. Fernandez et al., Academic Press, 1999; Molecular Cloning: A Laboratory Manual, Sambrook et al., 2nd ed., Cold Spring Harbor Laboratory Press, 1989; and Current Protocols in Molecular Biology, eds. Ausubel et al., 2nd ed., John Wiley & Sons, 1992.

TSHR agonists useful in the methods of the invention include small molecules. Small molecules include synthetic and purified naturally occurring TSHR agonists. Small molecules can be mimetics ore secretagogues. Examples of such molecules include Activators of Non-Genotropic Estrogen-Like Signaling (ANGELS) and related compounds (see, e.g., U.S. patent application Pub. No. 2003/0119800).

TSHR agonists useful in the methods of the invention further include inhibitors of thyroid hormone synthesis and/or release (e.g., small molecules such as propylthiouracil (PTU) and methimazole).

TSHR agonists useful in the method of the invention further include TSH secretagogues, such as, e.g., thyrotropin-releasing hormone (TRH; L-pyroglutamyl-L-histidyl-L-prolineamide).

Methods of Administration and Uses

The invention provides methods for treating or preventing bone degenerative disorders in mammals, including specifically humans, monkeys, rodents, sheep, rabbits, dogs, guinea pigs, horses, cows, and cats.

The disorders treated or prevented include, for example, osteopenia, osteomalacia, osteoporosis (e.g., post-menopausal, steroid-induced, senile, or thyroxin-use induced), osteomyeloma, osteodystrophy, Paget's disease, osteogenesis imperfecta, humoral hypercalcemic myeloma, multiple myeloma and bone thinning following metastatis. The disorders treated or prevented further include bone degenerative disorders associated with hypercalcemia, chronic renal disease, primary or secondary hyperparathyroidism, and long-term use of corticosteroids.

The disclosed methods include administering to a mammal a TSHR agonist in an amount effective to:

(1) treat or prevent a bone degenerative disorder;

(2) slow bone deterioration;

(3) restore lost bone;

(4) stimulate new bone formation; and/or

(5) maintain bone (bone mass and/or bone quality).

The methods of the invention can used to treat microdefects in trabecular and cortical bone. The bone quality can be determined, for example, by assessing microstructural integrity of the bone.

Generally, a TSHR agonist is administered repeatedly for a period of at least 2, 4, 6, 8, 10, 12, 20, or 40 weeks or for at least 1, 1.5, or 2 years or up to the life-time of the subject.

Generally, TSHR agonists, including thyrotropin and thyrotropin analogs, may be administered at a dose between 0.0001 and 0.001; 0.001 and 0.01; 0.01 and 0.1; or 0.1 and 10 lU/kg. In alternate embodiments, thyrotropin is administered at a dose (i) between 10⁻⁸ and 10⁻⁷; 10⁻⁷ and 10⁻⁶; 10⁻⁶ and 10⁻⁵; or 10⁻⁵ and 10⁻⁴ g/kg, wherein thyrotropin has specific activity between 0.01 and 100 lU/mg. In certain embodiments, the dose is not 7.2 lU/kg, 0.52 lU/kg, or 0.143 lU/kg, or the administration is not a single injection of up to 45 mg per human subject. As shown in the Examples, thyrotropin alfa can, for example, be injected intravenously for a 2-8 week period with doses of thyrotropin varying from 0.7 to 70 μg (corresponding to 0.005 and 0.5 lU, respectively). Therapeutically effective dosages achieved in one animal model can be converted for use in another animal, including humans, using conversion factors known in the art (see, e.g., Freireich et al. (1966) Cancer Chemother. Reports, 50(4):219-244).

The exact dosage of a TSHR agonist is determined empirically based on the desired outcome(s). Exemplary outcomes include: (a) bone degenerative disorder is treated or prevented, (b) bone deterioration is slowed; (c) lost bone is restored; (d) new bone growth is formed; and/or (e) bone mass and/or bone quality is maintained. For example, a TSHR agonist is administered in an amount effective to slow bone deterioration (e.g., loss of bone mass and/or bone mineral density) by at least 20, 30, 40, 50, 100, 200, 300, 400, or 500%.

The outcome(s) related to bone deterioration may also be evaluated by a specific effect of the TSHR agonists with respect to loss of trabecular bone (trabecular plate perforation); loss of (metaphyseal) cortical bone; loss of cancellous bone; decrease in bone mineral density, reduced bone mineral quality, reduced bone remodeling; increased level of serum alkaline phosphatase and acid phosphatase; bone fragility (increased rate of fractures), decreased fracture healing. Methods for evaluating bone mass and quality are known in the art and include, but are not limited to X-ray diffraction; DXA; DEQCT; pQCT, chemical analysis, density fractionation, histophotometry, and histochemical analysis as described, for example, in Lane et al. (2003) J. Bone Min. Res., 18(12):2105-2115 and in the Examples.

In some embodiments, compositions used in the methods of the invention further comprise a pharmaceutically acceptable excipient. As used herein, the phrase “pharmaceutically acceptable excipient” refers to any and all solvents, dispersion media, coatings, antibacterial and antifungal agents, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition is formulated to be compatible with its intended route of administration. For example, Thyrogen® is supplied as a sterile, non-pyrogenic, white to off-white lyophilized product, intended for intramuscular (IM) administration after reconstitution with Sterile Water for Injection, USP. Each vial of Thyrogen® contains 1.1 mg thyrotropin alfa (4-12 lU/mg), 36 mg mannitol, 5.1 mg sodium phosphate, and 2.4 mg sodium chloride. After reconstitution with 1.2 ml of Sterile Water for Injection, USP, the thyrotropin alfa concentration is 0.9 mg/ml. The pH of the reconstituted solution is approximately 7.0.

Alternatively, TSHR agonist may be provided in as described, for example, in Basu et al. (2004) Expert Opin. Biol. Ther., 4(3):301-317 and Pechenov et al. (2004) J. Control. Release, 96:149-158. Examples of such composition include crystalline protein formulations, provided naked or in combination with biodegradable polymers (e.g., PEG, PLGA).

Methods of administration are known in the art. “Administration” is not limited to any particular delivery system and may include, without limitation, parenteral (including subcutaneous, intravenous, intramedullary, intraarticular, intramuscular, or intraperitoneal injection) rectal, topical, transdermal, or oral (for example, in capsules, suspensions, or tablets). Administration to an individual may occur in a single dose or in continuous or intermittent repeat administrations, and in any of a variety of physiologically acceptable salt forms, and/or with an acceptable pharmaceutical carrier and/or additive as part of a pharmaceutical composition (described earlier). Physiologically acceptable salt forms and standard pharmaceutical formulation techniques and excipients are well known to persons skilled in the art (see, e.g., Physicians' Desk Reference (PDR) 2003, 57th ed., Medical Economics Company, 2002; and Remington: The Science and Practice of Pharmacy, eds. Gennado et al., 20th ed, Lippincott, Williams & Wilkins, 2000).

A TSHR agonist may be administered as a pharmaceutical composition in conjunction with carrier gels and matrices or other compositions used for guided bone regeneration and/or bone substitution. Examples of such matrices include synthetic polyethylene glycol (PEG)-, hydroxyapatite, collagen and fibrin-based matrices, tisseel fibrin glue, etc.

A TSHR agonist may be administered in combination or concomitantly with other therapeutic compounds such as, e.g., bisphosphonate (nitrogen-containing and non-nitrogen-containing), apomine, testosterone, estrogen, sodium fluoride, vitamin D and its analogs, calcitonin, calcium supplements, selective estrogen receptor modulators (SERMs, e.g., raloxifene), osteogenic proteins (e.g., BMP-2, BMP-4, BMP-7, BMP-11, GDF-8), statins, Activators of Non-Genotropic Estrogen-Like Signaling (ANGELS), and parathyroid hormone (PTH). Apomine is novel 1,1,-bisphosphonate ester, which activates farneion X activated receptor and accelerates degradation of HMG (3-hydroxy-3-methylglytaryl-coenzyme A) reductase (see, e.g., U.S. patent application Publication No. 2003/0036537 and references cited therein).

Administration of a therapeutic to an individual may also be by means of gene therapy, wherein a nucleic acid sequence encoding the antagonist is administered to the patient in vivo or to cells in vitro, which are then introduced into a patient. For specific gene therapy protocols, see Morgan, Gene Therapy Protocols, 2nd ed., Humana Press, 2000.

Additional applications of the present invention include use of TSHR agonists for coating, or incorporating into osteoimplants, matrices, and depot systems so as to promote osteointegration. Examples of such implants include dental implants and joint replacements implants.

The invention further comprises evaluating efficacy of a TSHR agonist for treatment of a bone degenerative disorder.

Such an assay comprises:

(1) administering the TSHR agonist repeatedly to a mammal (e.g., an OVX rat) for a period of at least 2, 4, 6, or 8 weeks; and

(2) determining the effect of the TSHR agonist on bone, wherein a slowing of bone deterioration (e.g., bone mass and/or bone quality) attributable to the TSHR agonist indicates that the TSHR agonist is effective for treatment of a bone degenerative disorder.

It will be understood that a TSHR agonist may be evaluated in one or more animal models of bone degenerative disorders and/or in humans. Osteopenia may be induced, for example, by immobilization, low calcium diet, high phosphorus diet, long term use of corticosteroid, cessation of ovary function, aging. For example, ovariectomy (OVX)-induced osteopenia is a well established animal model of human post-menopausal osteoporosis. Another well validated model involves administration of corticosteroids. Such models include: cynomolgus monkeys, dogs, mice, rabbits, ferrets, guinea pigs, minipigs, and sheep. For a review of various animal models of osteoporosis, see, e.g., Turner (2001) Eur. Cells and Materials, 1:66-81.

Additional in vitro tests may include evaluation of the effect on osteoblasts in culture such as the effect on collagen and osteocalcin synthesis or the effect on the level of alkaline phosphatase and cAMP induction. Appropriate in vivo and vitro tests are described in, for example, U.S. Pat. No. 6,333,312.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

### EXAMPLES

### Example 1

### Assays for Determining TSHR Agonists' Activity

Thyroid membrane preparation—The technique for preparing bovine thyroid membrane is based on a method described in Pekonen et al. (1980) J. Biol. Chem., 255:8121-8127. Calf thyroid glands are minced in 20 mM Tris HCl, 1 mM EDTA, pH 7.4. The minced tissue is homogenized in a single-speed VWR blender for 1 min and then filtered through cheesecloth. The homogenate is then centrifuged at 1000×g for 10 min at 4° C. and the pellet is discarded. The supernatant is removed and centrifuged at 10 000×g for 30 min at 4° C. The resulting pellet is resuspended in 20 mM Tris HCl, 1 mM EDTA, pH 7.4 and then centrifuged again at 10 000×g for 30 min. The pellet was resuspended and centrifuged at 18 000×g for 30 min. This may be repeated once more and, after quantification using BioRad Protein Assay according to the manufacturer's instructions, the pellet is resuspended to a final concentration of 1.25 mg/ml in 0.25 M sucrose, 20 mM Tris HCl, 1 mM EDTA, pH 7.4. The thyroid preparation is pulsed briefly in the blender, aliquotted, and then stored at less than −70° C.

Membrane-based TSH specific activity assay—Reference and test samples are analyzed at three levels. Twenty μl of the bovine thyroid membrane preparation (described above) are added to 80 μl of the sample which contains 20 ng, 60 ng, or 180 ng of TSH in 1.25 mM EDTA, 0.125 mM BSA, 25 μl theophylline, 62.5 μM 5′-guanylyl-imidodiphosphate. 17.4 mM creatine phosphate, 12.5 mM creatine phosphokinase, 2.5 mM ATP, 6.25 mM magnesium chloride, 25 mM Tris HCl, pH 7.8. Samples are vortexed, incubated at 30° C. for 20 min, boiled for 5 min, and then cooled in ice water for 15 min. Cyclic AMP is quantified by RIA according to the manufacturer's recommendations (NEN).

Growth and maintenance of TSH-responsive cell line—A CHO cell line that is stably transfected with the TSH receptor and a cAMP-responsive luciferase reporter is obtained from Interthyr Corporation (Athens, Ohio, U.S.A.). Cells were cultured in Ham's F-12 medium containing 10% FBS, 2 mM L-glutamine, 100 units penicillin and 100 μg streptomycin per ml. Cultures is grown in a humidified cell incubator under a 5% CO, atmosphere. Cells are subcultured when they reached 90-100% confluency.

Cell-based TSH potency assay—Cells are seeded at 30 000 cells/well in growth medium in a 96-well tissue culture plate (Costar), excluding outside wells, to which medium is added. Plates were incubated 17-19 h in a humidified 5% CO, incubator. The growth medium is replaced with 0.4% BSA in Hanks' Balanced Salt Solution (HBSS) containing 0 to 60 pg/ml rhTSH. A nominal specific activity of 5.3 lU/mg is assigned to this reference material which is tested against a human TSH reference standard (WHO 841703, National Institute for Biological Standards and Controls, Potters Bar, U.K.) using the membrane-based specific activity assay described above. Positive (60 μg/ml rhTSH) and negative (0 μg/ml rhTSH) controls are tested in six wells, and the reference and sample curves span the range of 20 μg/ml to 0.0012 μg/ml rhTSH. Each level of reference and sample is tested in triplicate wells. The plates are incubated in a humidified 5% CO₂ incubator for 6 hour. Intracellular luciferase activity is determined using the Luciferase Assay Reagent Kit (Promega, Madison, Wis., U.S.A.) according to the manufacturer's recommendations. Luminescence was measured on a Wallac Microlumat Plus LB 96 V luminometer.

### Example 2A

### Treatment of Osteopenia in Rats

Seventy two 4 months old Sprague-Dawley female rats, weighting approximately 300 g were used in this study. The rats were kept in standard conditions (24° C. and 12 h/12 h light-dark cycle) in 20×32×20 cm cages during experiment. All animals had allowed free access to water and pelleted commercial diet (Harlan Teklad) containing 1,00% calcium, 0,65% phosphorus and 2,40 KlU of Vitamin D3 per kilogram. Animals received on days −14 and −4 calcein green labeling regimen (15 mg/kg i.p.), which resulted in the deposition of double fluorochrome labels on active bone forming surfaces.

Twelve animals were sham operated, while sixty were ovariectomized (OVX) bilaterally by abdominal approach. Treatment started immediately after ovariectomy as follows: (1) SHAM; (2) OVX+vehicle daily; (3) OVX+0.7 μg thyrotropin daily; (4) OVX+7 μg thyrotropin; (5) OVX+70 μg thyrotropin daily; (6) OVX+17β-estradiol 3 times a week. Animals were treated for 8 weeks (before DEXA analysis). Thyrogen® used in this study had specific activity of 7 lU/mg.

Animals were scanned at the beginning of therapy and than every two weeks during eight weeks of therapy using dual energy absorptiometry (DXA, HOLOGIC QDR-4000) equipped with Small Animal software. Prior to scan animals were anaesthetized with Thiopental (Nycomed).

Total body scans were performed and bone mineral density (BMD), bone mineral content (BMC) of lumbar vertebrae, of hind limbs, total body, and total body with head excluded were determined.

For urine collection animals were placed in metabolic cages and deprived of food for an overnight period of 18 hours. Blood was taken from orbital plexus. Blood and urine was collected at the beginning of therapy and than every two weeks during eight weeks of therapy.

Sacrifice started 8 weeks after the beginning of therapy in ether anesthesia. Bones were collected for histology.

After sacrifice, femora, tibiae and lumbar vertebrae were harvested and scanned, and BMD and BMC of whole bone and its parts were measured

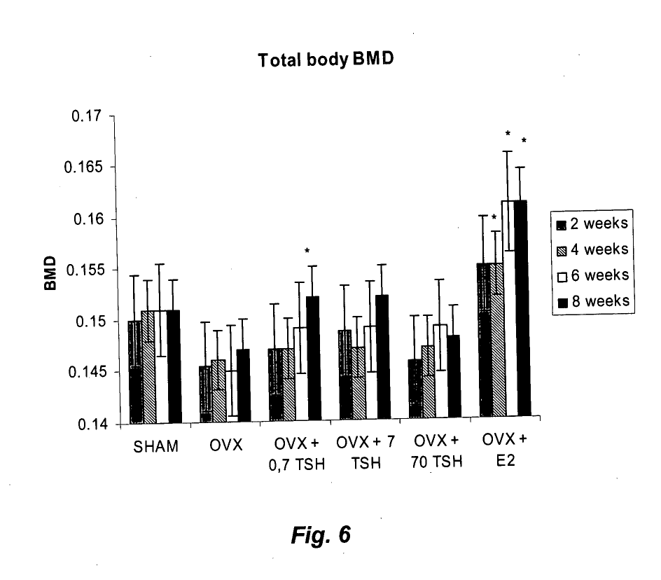

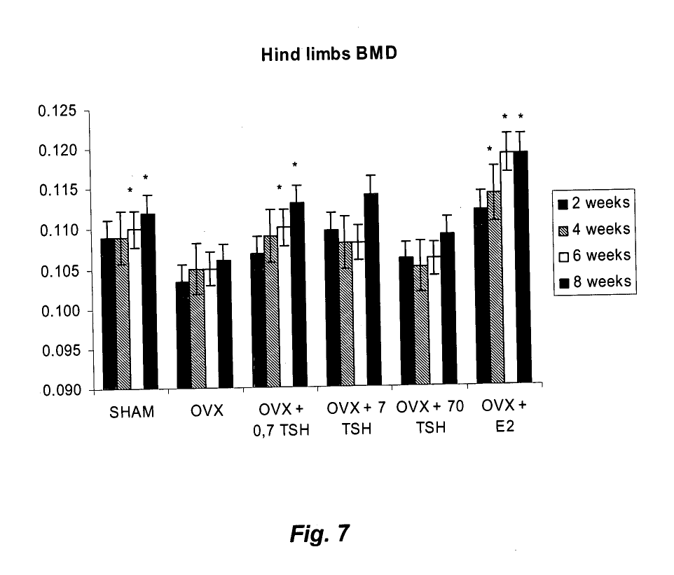

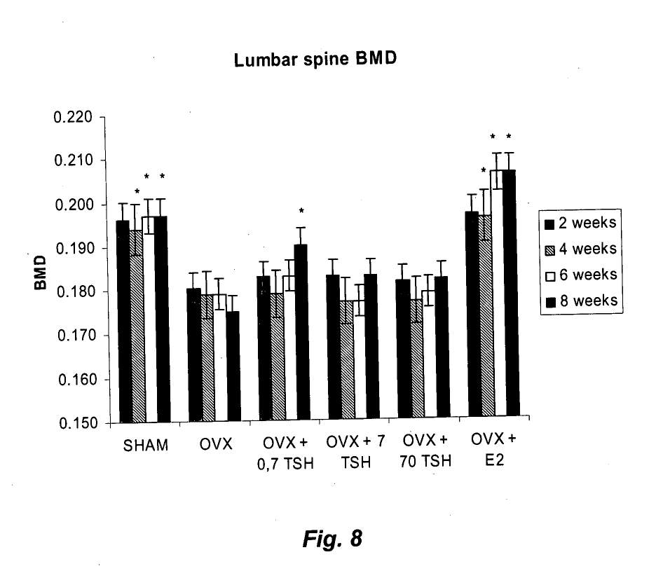

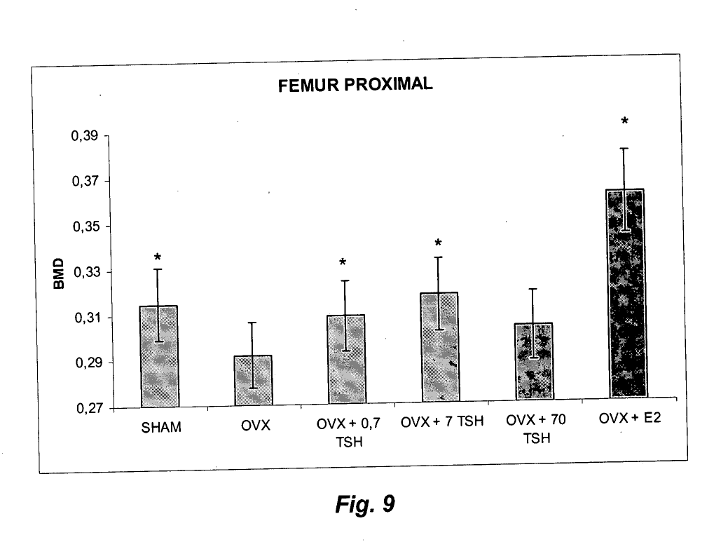

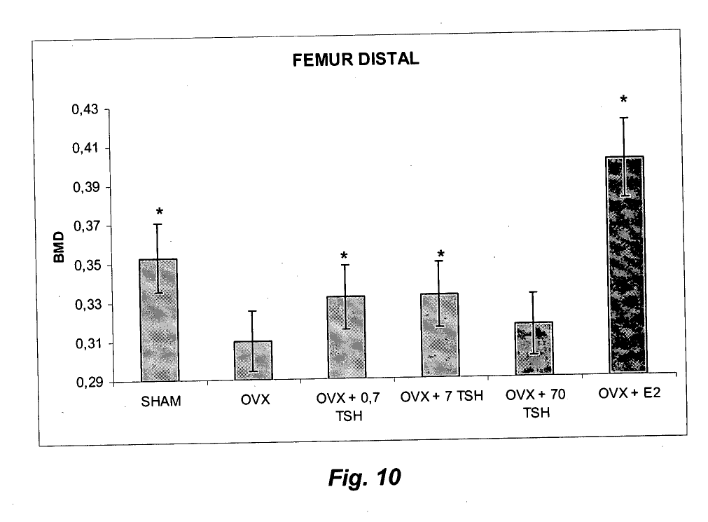

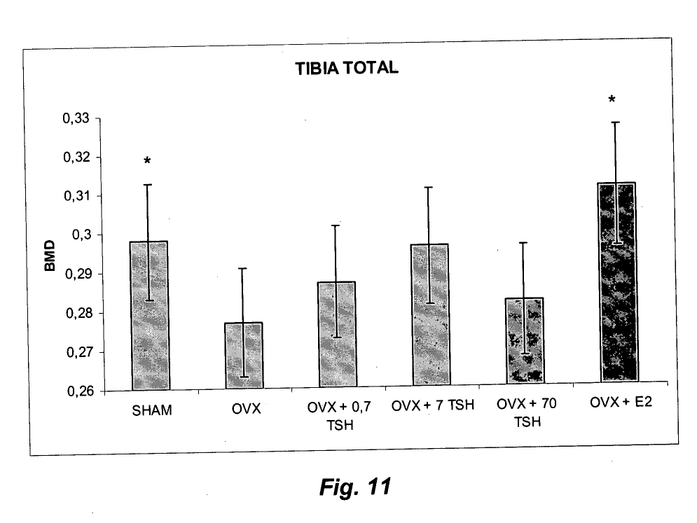

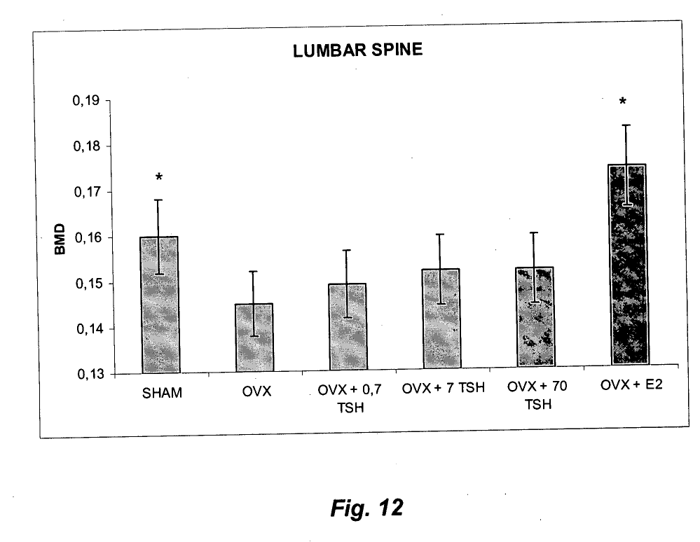

FIGS. 6-8 show the results of BMD analysis at 2, 4, 6, and 8 weeks for total body, hind limbs, and lumbar, respectively. The BMD analysis of hind limbs (including hip) revealed that Thyrogen® at lower doses (0.7 μg/rat) increased BMD at weeks 6 and 8 as compared to OVX at weeks 2 and 4, while reaching a statistically significant level at 6 weeks. Thyrogen® is also able to influence vertebrae BMD positively at 6 weeks. At higher doses (70 μg/rat), Thyrogen® appears to be more resorptive than anabolic. Similarly to parathyroid hormone (PTH), it is likely that Thyrogen®) may be exhibiting a biphasic action on bone remodeling. FIGS. 9-12 show results of ex vivo DEXA measurements of bones. The results demonstrate a statistically significant increase in BMD of proximal and distal femur in animals treated with 0.7 and 7 μg of human thyrotropin as compared to OVX animals. BMD of tibia and lumbar spine showed a trend but no statistical differences between thyrotropin treated groups and OVX animals. Ex vivo DEXA measurements showed that BMD of proximal femur reached sham values in animals treated with 0.7 and 7 μg of human thyrotropin. There were no significant differences in body weight between experimental groups and no observed side-effects.

### Example 2B

### Treatment of Osteopenia in Rats Using Rat TSH

A similar study following essentially the same dosing regimens. was performed using the native rat TSH in OVX rats. As rat TSH issignificantly more effective (10-20 times) as compared to human recombinant TSH in stimulating thyroid hormone production by the thyroid in rats, the doses 0.01, 0.1, 0.3 μg per rat were used. Rat TSH with a specific activity of approximately 90 lU/mg was obtained from the Scripps Institute. Bone mineral monitoring in vivo and serum and urine biochemical analyses were performed essentially as described in Example 3A. The results of this study demonstrated that native rat TSH is effective in preventing the bone loss associated with ovariectomy as determined by in vivo analyses of total body, hind limbs and lumbar BMD and ex vivo analyses of proximal femur and proximal tibia BMD, and trabecular bone volume, trabecular number, trabecular thickness, cortical thickness and bone mineral content, as determined by microCT analyses. In addition, reduction in serum collagen C-telopeptide analysis indicated that TSH has anti-resorptive activity. As expected, rat TSH was effective in the rat at lower concentration than human TSH.

### Example 3A

### In vivo Characterization of Treatment Effects

Biochemical assays—Urinary levels of deoxypyridinoline cross-links and creatinine (DPD and Cr, respectively) are analyzed in duplicate using rat ELISA kits from Metrobiosystems (Mountain View, Calif., USA). Serum levels of osteocalcin (OSC) are measured using a rat sandwich ELISA kit from Biomedical Technologies (Stroughton, Mass., USA). The manufacturer's protocols are followed, and all samples are assayed in duplicate. A standard curve is generated from each kit, and the absolute concentrations are extrapolated from the standard curve.

The right proximal tibial metaphyses are imaged without further sample preparation with a desktop μCT (μCT20; Scanco Medical, Bassersdorf, Switzerland), with a resolution of 26 μm in all three spatial dimensions (Laib et al. (2001) Osteoporos. Int., 12:936-941). The scans are initiated from the growth plate distally in 26-μm sections, for a total of 120 slices per scan. From this region, 60 slices starting at a distance of 1 mm distal from the lower end of the growth plate and encompassing a volume of 1.56 mm length are chosen for the evaluation. The trabecular and the cortical regions are separated with semiautomatically drawn contours.

The complete secondary spongiosa of the proximal tibia is evaluated, thereby completely avoiding sampling errors incurred by random deviations of a single section. The resulting gray-scale images are segmented using a lowpass filter to remove noise, and a fixed threshold is used to extract the mineralized bone phase. From the binarized images, structural indices are assessed with three-dimensional (3D) techniques for trabecular bone.

Relative bone volume, trabecular number, thickness, and separation are calculated by measuring 3D distances directly in the trabecular network and taking the mean over all voxels.

Bone surface is calculated from a tetrahedron meshing technique. By displacing the surface of the structure in infinitesimal amounts, the structure model index (SMI) is calculated. The SMI quantifies the plate versus rod characteristics of trabecular bone, in which an SMI of 0 pertains to a purely plate-shaped bone, an SMI of 3 designates a purely rod-like bone, and values between stand for mixtures of plates and rods. Furthermore, connectivity density based on the Euler number is determined. In addition, a 3D cubical voxel model of bone is built, and cortical thickness is measured.

Bone histomorphometry—The right proximal tibias are dehydrated in ethanol, embedded undecalcified in methylmethacrylate, and sectioned longitudinally with a Leica/Jung 2065 microtome in 4- and 8-μm-thick sections. The 4-μm sections are stained with von Kossa and Toluidine blue for collection of bone mass and architecture data with the light microscope, whereas the 8-μm sections are left unstained for measurements of fluorochrome-based indices. Static and dynamic histomorphometry are performed using a semi-automatic image analysis OsteoMeasure System (OsteoMetrics Inc., Decatur, Ga., USA) linked to a microscope equipped with transmitted and fluorescence light or by Skeletech (Seattle, Wash., USA).

A counting window, measuring 8 mm² and containing only cancellous bone and bone marrow, is created for the histomorphometric analysis. Static measurements included total tissue area, bone area, and bone perimeter. Dynamic measurements included single and double-labeled perimeter, osteoid perimeter, and interlabel width. These indices are used to calculate bone volume, trabecular number, trabecular thickness and trabecular separation, osteoid surface, mineralizing surface, and mineral apposition rate (MAR). Osteoid volume is measured separately and is not included in the volume for cancellous bone. Mineralization lag time in days (MLT) is calculated as osteoid thickness/MAR. Finally, surface-based bone formation rate (BFRBS) is calculated by multiplying mineralizing surface (single-labeled surface/2+double-labeled surface) with MAR.

Mechanical property testing—For topographic imaging and discrete mechanical properties determination of individual trabeculae, a modified atomic force microscope (AFM; Nanoscope IIIa; Digital Instruments, Santa Barbara, Calif., USA) is used. The modification consisted of replacing the cantilever/tip assembly of the microscope with a transducer driven head and tip (Triboscope; Hysitron, Minneapolis, Minn., USA) that allowed the microscope to operate both as an imaging and an indentation instrument. The detailed modifications for this discrete indentation have been described in detail elsewhere. All indentations are performed with a triangular load profile of 0.3 mm/s in time to 300-μN maximum load. Elastic modulus and hardness are calculated from the unloading force/displacement slope at maximum load and the projected contact area at this load. The instrument is then further modified to perform dynamic stiffness imaging that allows simultaneous determination of surface topography and both storage and loss moduli by applying a small sinusoidal force on the AFM tip in contact mode and measuring the resulting displacement amplitude and its phase lag with respect the force. These quantities are used to determine the viscoelastic properties, pixel-by-pixel, as the tip scanned over the surface of the bone. In the present work, the loss modulus is found to be less than 5% of storage modulus; therefore, we considered the storage modulus to be roughly equivalent to the elastic modulus (small viscoelastic effect).

The methylmetacrylate-embedded right proximal tibial metaphyses samples (approximately 3 mm thick) that had been used for bone histomorphometry are further polished on one side with progressively finer grades of diamond paste (down to 0.1 μm) until a smooth bone surface is exposed (approximate nanometer roughness). The AFM measurements are performed on different trabeculae on each specimen in both longitudinal as well as transverse orientations. Three right proximal tibial metaphyseal bone samples from each of the four treatment groups (sham, OVX, and TSH) are tested (approximately 20 trabeculae per bone specimen). The elastic modulus and hardness are obtained by indentation along a line crossing the edge of the samples with an interval of 2 mm, covering a length of at least 30 mm for each trabeculae measured.

### Example 3B

### Bone Restoration Study in Rats

To confirm that TSH has an anabolic effect on bone, a restoration study was performed, Native rat TSH was administered seven months after ovariectomy essentially following a similar dose and dosing regimen as described in Example 2B except that administration of TSH was extended until 16 weeks.

Twelve animals were sham operated, while forty eight were ovariectomized (OVX) bilaterally by abdominal approach. Treatment started six weeks after ovariectomy as follows: (1) SHAM (n=12); (2) OVX+vehicle (n=12); (3) OVX+0.01 μg (n=12); (3) OVX+0.1 μg (n=12); and (4) OVX+0.3 μg (n=12).

Bone mineral density monitoring in vivo and ex vivo and serum and urine biochemical analyses were performed essentially as described in Example 3A. Eight-month old rats were ovariectomized and then left for seven months to lose bone. Therapy was then started and continued for 16 weeks and BMD was measured both in vivo and ex vivo, and microCT and serum biochemistry were performed.

The results showed the following:

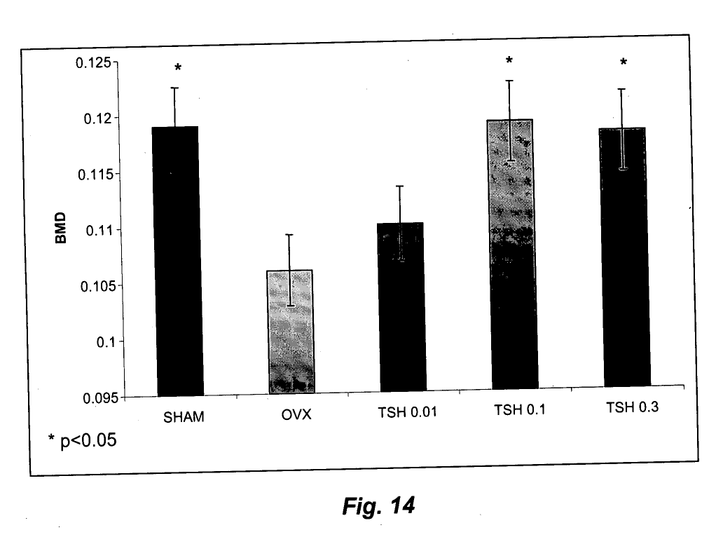

(1) TSH increased BMD values of hind limbs in vivo at concentrations of 0.1 and 0.3 μg/rat, while the lowest tested dose of 0.01 μg/rat did not induce a measurable effect at 12 and 16 weeks time points (FIG. 14);

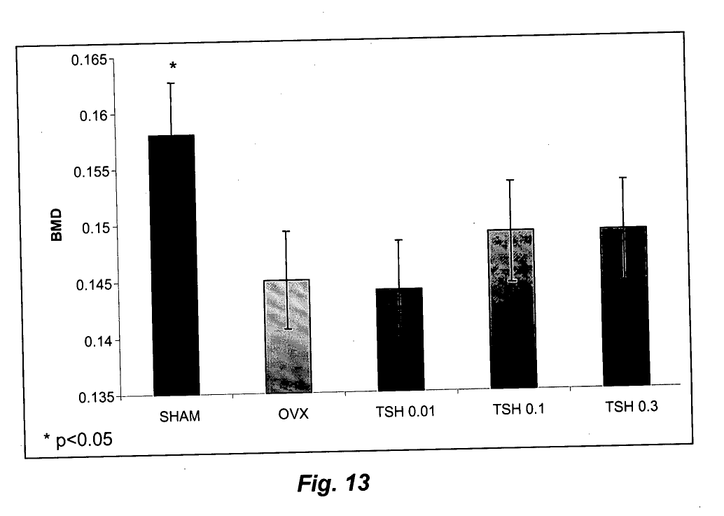

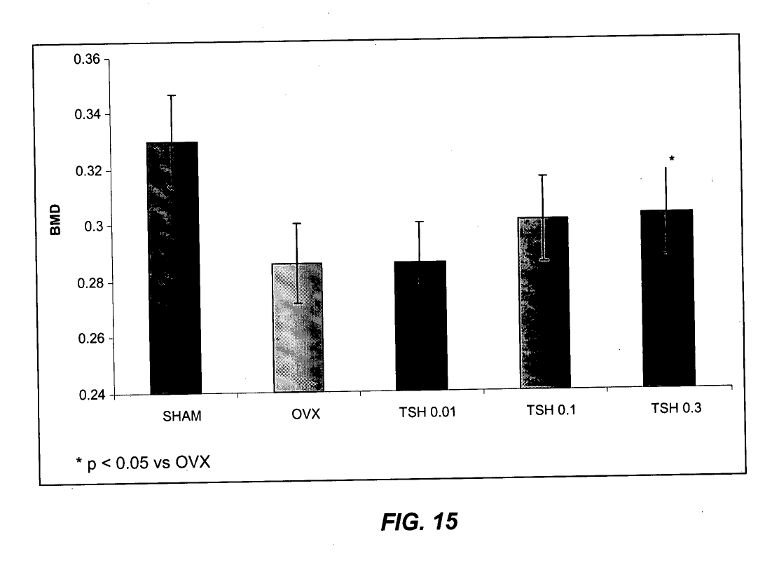

(2) ex vivo proximal femur BMD was increased with a dose of 0.3 μg and distal and total femur BMD values were slightly increased as measured by DEXA (FIGS. 13 and 15);

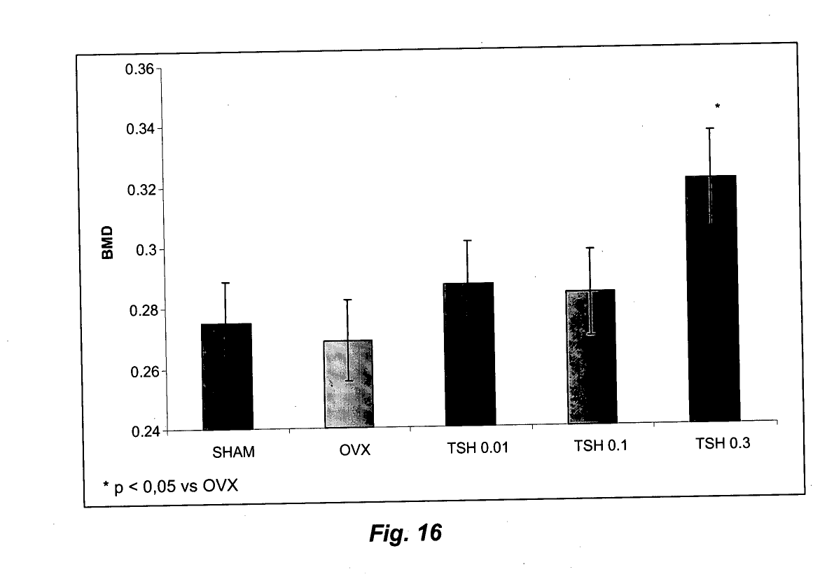

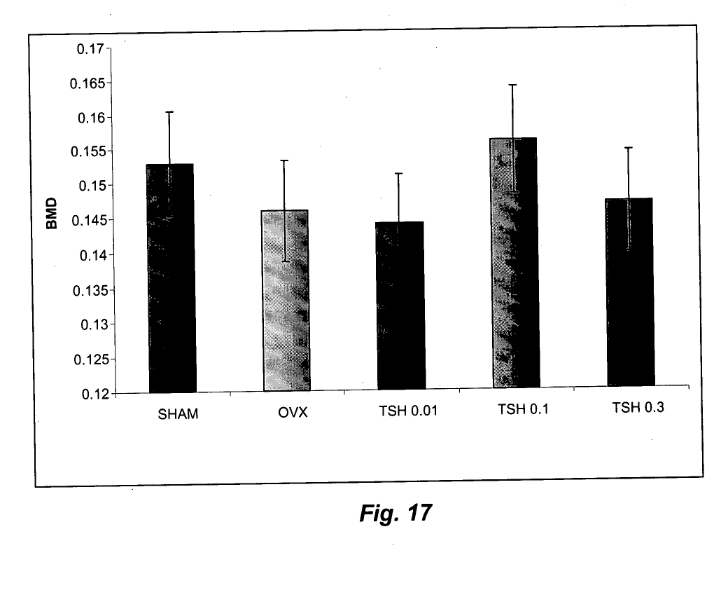

(3) ex vivo tibia and lumbar spine BMD values were also increased, in particular, with 0.1 and 0.3 μg/rat doses (FIGS. 16 and 17);

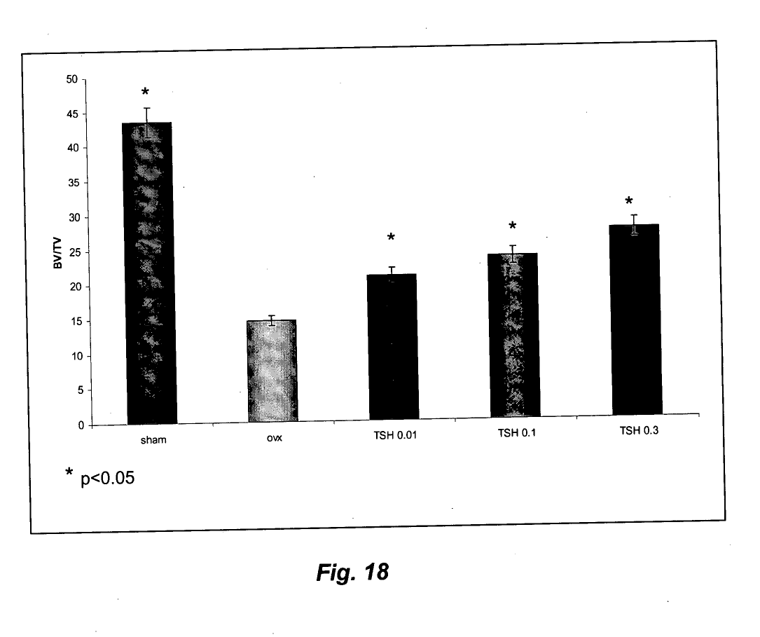

(4) microCT analyses of trabecular bone ex vivo revealed that the bone volume of long bones and the spine was increased in animals treated with all three doses tested (FIG. 18);

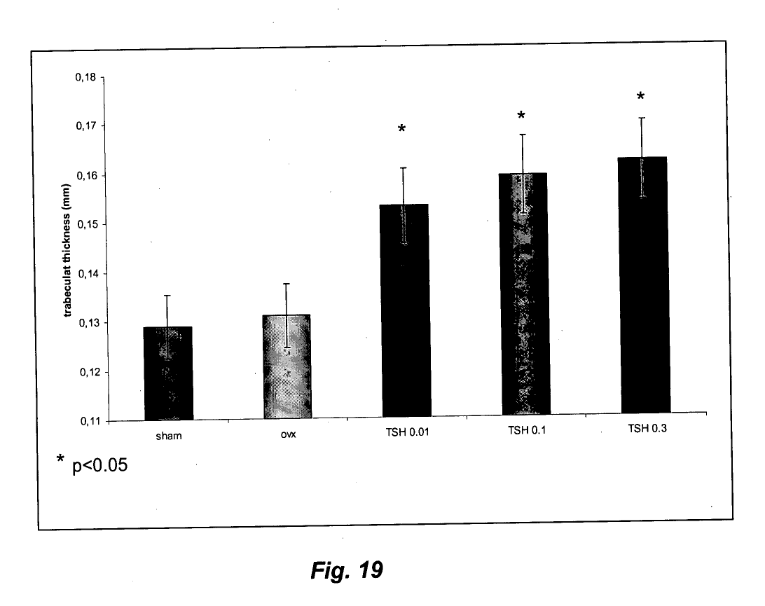

(4) trabecular thickness (measured by microCT) was significantly increased above levels of sham and ovariectomized rats (FIG. 19);

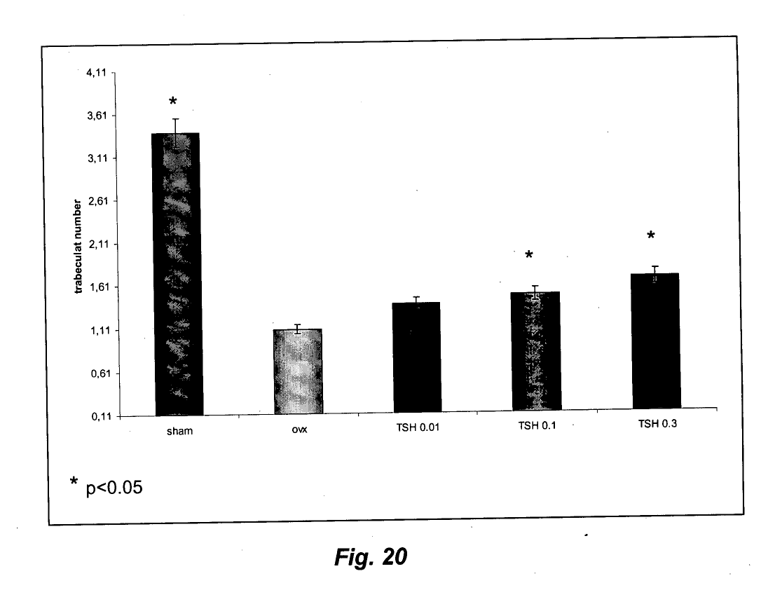

(5) trabecular number was also increased as measured by microCT but to a lesser extent then trabecular thickness due to aged animals (FIG. 20);

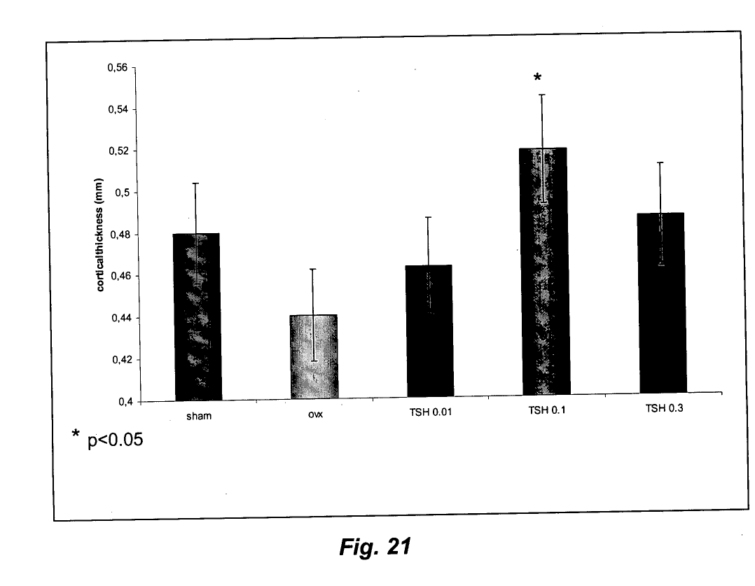

(6) both doses of 0.1 and 0.3 μg/rat increased the cortical thickness with 0.1 μg/rat dose being almost 10% above sham animal values, and 14% above ovariectomized rats (FIG. 21); and

(7) there was no measurable increase of T3 and T4 serum levels (not shown).

These results were consistent with another study performed, where rat TSH at 0.1 μg/rat was used in ovariectomized rats.

### Example 4

### Treatment of Humans with a TSHR Agonist

This Example describes a prospective clinical trial for treatment of osteoporosis with a TSHR agonist in humans. Subjects will be selected from postmenopausal women with either normal thyroid function or with thyroid dysfunction exhibiting low circulating thyrotropin with prior vertebral fractures (more than one) who have been treated previously with Fosomax® (alendronate) or SERM (raloxifene). A TSHR agonist, e.g., thyrotropin or its analogues will be administered (e.g., daily, weekly, or biweekly) systemically (e.g., intravenous, subcutaneous, intramuscular, oral, or transdermal routes). Subjects will receive have Dose I (low) or Dose II (high) of the TSHR antagonist or placebo, which will be made available systemically.

Vertebral radiographs at base line and by the end of the study (median duration of observation, 24 months) will be performed. Serial measurements of bone mass by dual-energy x-ray absorptiometry (BMD) at 6 months intervals will be performed. Biochemical markers for bone formation and bone resorption will be determined in blood and urine at 3-6 months intervals. The subjects will be monitored for subsequent fractures (both vertebral and non-vertebral), if any, during the completion of the study.

Treatment of postmenopausal osteoporosis with Thyrogen® is expected to decrease the risk of vertebral and non-vertebral fractures, to increase vertebral, femoral, and total-body bone mineral density, and to be well tolerated.

Data will be analyzed for all women with at least one follow-up visit after enrollment. The rates of side effects and the proportions of women with fractures in the three study groups will be compared with the use of Pearson's chi-square test. All laboratory data and bone mineral measurements will be evaluated by analysis of variance, with the inclusion of terms for the treatment assignment and country. The statistical tests will be two-sided.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and biological sequences cited in this disclosure are incorporated by reference in their entirety. To the extent the material incorporated by reference contradicts or is inconsistent with the present specification, the present specification will supersede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

1. A method of treating or preventing a bone degenerative disorder in a mammal, the method comprising administering thyrotropin to the mammal in an amount and for a period of time sufficient to treat or prevent the bone degenerative disorder.

2. The method of claim 1, wherein the bone degenerative disorder is chosen from osteopenia, osteomalacia, osteoporosis, osteomyeloma, osteodystrophy, Paget's disease, osteogenesis imperfecta, bone sclerosis, aplastic bone disorder, humoral hypercalcemic myeloma, multiple myeloma, and bone thinning following metastasis.

3. The method of claim 2, wherein the disorder is osteoporosis.

4. The method of claim 2, wherein osteoporosis is post-menopausal, steroid-induced, senile, or thyroxin-use induced.

5. The method of claim 1, wherein the bone degenerative disorder in the mammal is associated with one or more of: hypercalcemia, chronic renal disease, kidney dialysis, primary and secondary hyperparathyroidism, and long-term use of corticosteroids.

6. A method of slowing bone deterioration, maintaining bone, restoring lost bone, or stimulating new bone formation in a mammal, the method comprising administering a therapeutically effective amount of thyrotropin to the mammal for a period of time sufficient to slow bone deterioration, maintain bone, restore lost bone, or stimulate new bone formation.

7. The method of claim 6, wherein the bone deterioration is characterized by a loss of bone mass.

8. The method of claim 7, wherein the loss of bone mass is determined by measuring bone mineral density.

9. The method of claim 6, wherein the bone deterioration is characterized by degeneration of bone quality.

10. The method of claim 9, wherein degeneration of bone quality is determined by assessing microstructural integrity of the bone.

11. The method of claim 1 or 6, further comprising administering a second therapeutic compound selected from the group consisting of: bisphosphonate, bisphosphonate ester, testosterone, estrogen, sodium fluoride, vitamin D and its analogs, calcitonin, a calcium supplement, a selective estrogen receptor modulator, osteogenic protein, statin, ANGELS, and PTH.

12. The method of claim 1 or 6, wherein the mammal is human.

13. The method of claim 1 or 6, wherein the thyrotropin is recombinant thyrotropin.

14. The method of claim 1 or 6, wherein the recombinant thyrotropin is produced in CHO cells.

15. The method of claim 1 or 6, wherein thyrotropin is human.

16. The method of claim 15, wherein the human thyrotropin is thyrotropin alpha.

17. The method of claim 1 or 6, wherein thyrotropin comprises a sequence as set out from amino acid 1 to amino acid 112 of SEQ ID NO:3.

18. The method of claim 1 or 6, wherein thyrotropin comprises a sequence as set out from amino acid 1 to amino acid 118 of SEQ ID NO:3.

19. The method of claim 1 or 6, wherein thyrotropin further comprises a sequence as set out in SEQ ID NO:1.

20. The method of claim 1 or 6, wherein thyrotropin is administered at a dose between 0.0001 and 0.01; 0.01 and 0.1; or 0.1 and 10 lU/kg.

21. The method of claim 1 or 6, wherein thyrotropin is administered systemically at a dose between 10⁻⁸ and 10⁻⁷, 10⁻⁷ and 10⁻⁶; 10⁻⁶ and 10⁻⁵, or 10⁻⁵ and 10⁻⁴ g/kg, wherein thyrotropin has specific activity between 0.01 and 100 lU/mg.

22. The method of claim 1 or 6, wherein thyrotropin is administered systemically.

23. The method of claim 1 or 6, wherein thyrotropin is administered repeatedly over a period of time of at least 2 weeks.


SEQUENCE LISTING (66 sequences)

SEQ ID NO: 1 - protein, 92 aa, Human
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 2 - DNA, 351 bp, Human
atggattactacagaaaatatgcagctatctttctggtcacattgtcggtgtttctgcatgttctccattccgctcctgatgtgcaggattgcccagaatgcacgctacaggaaaacccattcttctcccagccgggtgccccaatacttcagtgcatgggctgctgcttctctagagcatatcccactccactaaggtccaagaagacgatgttggtccaaaagaacgtcacctcagagtccacttgctgtgtagctaaatcatataacagggtcacagtaatggggggtttcaaagtggagaaccacacggcgtgccactgcagtacttgttattatcacaaatcttaa

SEQ ID NO: 3 - protein, 118 aa, Human
FCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSYLVGFSV

SEQ ID NO: 4 - DNA, 417 bp, Human
atgactgctctctttctgatgtccatgctttttggccttgcatgtgggcaagcgatgtctttttgtattccaactgagtatacaatgcacatcgaaaggagagagtgtgcttattgcctaaccatcaacaccaccatctgtgctggatattgtatgacacgggatatcaatggcaaactgtttcttcccaaatatgctctgtcccaggatgtttgcacatatagagacttcatctacaggactgtagaaataccaggatgcccactccatgttgctccctatttttcctatcctgttgctttaagctgtaagtgtggcaagtgcaatactgactatagtgactgcatacatgaagccatcaagacaaactactgtaccaaacctcagaagtcttatctggtaggattttctgtctaa

SEQ ID NO: 5 - protein, 18 aa, Artificial sequence
  Hypothetical sequence
CXXXXNXXFSXXXXPXXC

SEQ ID NO: 6 - protein, 19 aa, Human
CTLQENPFFSQPGAPILQC

SEQ ID NO: 7 - protein, 19 aa, Baboon
CKPRENQFFSQPGAPILQC

SEQ ID NO: 8 - protein, 19 aa, Bass
CTLRKNSVFSRDRAPVYQC

SEQ ID NO: 9 - protein, 19 aa, Bovine
CKLKENKYFSKPDAPIYQC

SEQ ID NO: 10 - protein, 19 aa, Carp
CKLKENNIFSKPGAPVYQC

SEQ ID NO: 11 - protein, 19 aa, Catfish
CKLKENNIFSKPGAPVYQC

SEQ ID NO: 12 - protein, 19 aa, Chicken
CKLGENRFFSKPGAPIYQC

SEQ ID NO: 13 - protein, 19 aa, Chimpanzee
CTLQENPFFSQPGAPILQC

SEQ ID NO: 14 - protein, 19 aa, Dog
CKLKENKYFSKLGAPIYQC

SEQ ID NO: 15 - protein, 19 aa, Donkey
CKLKKNKYFSKLGVPIYQC

SEQ ID NO: 16 - protein, 19 aa, Eel (Europ.)
CRLQENKIFSKPSAPIFQC

SEQ ID NO: 17 - protein, 19 aa, Eel (pike)
CRLKDNKFFSKPSAPIFQC

SEQ ID NO: 18 - protein, 19 aa, Gerbil
CKLKENKYFSKGGAPIYQC

SEQ ID NO: 19 - protein, 19 aa, Gibbon
CQLHENPFFSQPGAPILQC

SEQ ID NO: 20 - protein, 19 aa, Goldfish
CKLKENNIFSKPGAPVYQC

SEQ ID NO: 21 - protein, 19 aa, Guinea pig
CKLKENKLFSMLGAPIYQC

SEQ ID NO: 22 - protein, 19 aa, Hamster
CKLKENKYFSKLGAPIYQC

SEQ ID NO: 23 - protein, 19 aa, Horse
CKLRENKYFFKLGVPIYQC

SEQ ID NO: 24 - protein, 19 aa, Kangaroo
CKLKENKYFSKLGAPIYQC

SEQ ID NO: 25 - protein, 19 aa, Lungfish
CKLKENKYFSKPGAPIYQC

SEQ ID NO: 26 - protein, 19 aa, Macaque
CKPRENKFFSKPGAPIYQC

SEQ ID NO: 27 - protein, 19 aa, Marmoset
CKLKENKYFSRLGSPIYQC

SEQ ID NO: 28 - protein, 19 aa, Mouse
CKLKENKYFSKLGAPIYQC

SEQ ID NO: 29 - protein, 19 aa, Orangutan
CTLQENPFFSQPGAPILQC

SEQ ID NO: 30 - protein, 19 aa, Ostrich
CKLGENRFFSKPGAPVYQC

SEQ ID NO: 31 - protein, 19 aa, Ovine
CKLKENKYFSKPDAPIYQC

SEQ ID NO: 32 - protein, 19 aa, Porcine
CKLKENKYFSKLGAPIYQC

SEQ ID NO: 33 - protein, 19 aa, Quail
CKLGENRFFSKPGAPIYQC

SEQ ID NO: 34 - protein, 19 aa, Rabbit
CKLKENKYFSKLGAPIYQC

SEQ ID NO: 35 - protein, 19 aa, Rat
CKLKENKYFSKLGAPIYQC

SEQ ID NO: 36 - protein, 19 aa, Salmon
CKLKENKVFSNPGAPVYQC

SEQ ID NO: 37 - protein, 19 aa, Seabream
CTLRKNTVFSRDRAPIYQC

SEQ ID NO: 38 - protein, 19 aa, Tilapia
CTLRKNNLFSRDRAPVYQC

SEQ ID NO: 39 - protein, 19 aa, Tuna
CTLKKNNVFSRDRAPIYQC

SEQ ID NO: 40 - protein, 19 aa, Turkey
CKLGENRFFSKPGAPIYQC

SEQ ID NO: 41 - protein, 19 aa, Whale
CKLKQNKYFSKLGAPIYQC

SEQ ID NO: 42 - protein, 19 aa, Yellowfin
CTLRKNTVFSRDRAPIYQC

SEQ ID NO: 43 - protein, 19 aa, Zebra
CKLKVNKYFSKLGVPIYQC

SEQ ID NO: 44 - protein, 96 aa, Generic
  misc_feature (4)..(4): G, absent, or any other amino acid
XPDXXXXXXXCPECKCQENXXFSXXGXPIXQCMGCCFSRAYPTPARSKKTMLVPKNXTSEXTCCVAKSYTRVTVMXXXXXENHTXCXCSTCYXXKX

SEQ ID NO: 45 - protein, 92 aa, Homo sapiens; identical to SEQ ID NO: 1

SEQ ID NO: 46 - protein, 96 aa, Macaca mulatta
FPDGEFTMQDCPECKPRENKFFSKPGAPIYQCMGCCFSRAYPTPVRSKKTMLVQKNVTSESTCCVAKSLTRVMVMGSVRVENHTECHCSTCYYHKF

SEQ ID NO: 47 - protein, 96 aa, Callithrix jacchus
LPDGEFTAEECPECKLKENKYFSRLGSPIYQCMGCCFSRAYPTPLRSQKTMLVPKNVTSESTCCVAKAYTKATVMGNIRVENHTECHCSTCYHHKF

SEQ ID NO: 48 - protein, 96 aa, Bos taurus
FPDGEFTMQGCPECKLKENKYFSKPDAPIYQCMGCCFSRAYPTPARSKKTMLVPKNITSEATCCVAKAFTKATVMGNVRVENHTECHCSTCYYHKS

SEQ ID NO: 49 - protein, 96 aa, Ovis aries; identical to SEQ ID NO: 48

SEQ ID NO: 50 - protein, 96 aa, Sus scrofa
FPDGEFTMQGCPECKLKENKYFSKLGAPIYQCMGCCFSRAYPTPARSKKTMLVPKNITSEATCCVAKAFTKATVMGNARVENHTECHCSTCYYHKS

SEQ ID NO: 51 - protein, 96 aa, Equus caballus
FPDGEFTTQDCPECKLRENKYFFKLGVPIYQCKGCCFSRAYPTPARSRKTMLVPKNITSESTCCVAKAFIRVTVMGNIKLENHTQCYCSTCYHHKI

SEQ ID NO: 52 - protein, 96 aa, Equus asinus
FPDGEFTTQDCPECKLKKNKYFSKLGVPIYQCMGCCFSRAYPTPARSKKTMLVPKNITSEATCCVAKAFIRVTLMGNIRLENHTQCYCSTCYHHKI

SEQ ID NO: 53 - protein, 96 aa, Oryctolagus cuniculus
FPDGEFAMQGCPECKLKENKYFSKLGAPIYQCMGCCFSRAYPTPARSKKTMLVPKNITSEATCCVAKAFTKATVMGNAKVENHTECHCSTCYYHKS

SEQ ID NO: 54 - protein, 96 aa, Rattus norvegicus
LPDGDFIIQGCPECKLKENKYFSKLGAPIYQCMGCCFSRAYPTPARSKKTMLVPKNITSEATCCVAKAFTKATVMGNARVENHTECHCSTCYYHKS

SEQ ID NO: 55 - protein, 96 aa, Mus musculus; identical to SEQ ID NO: 54

SEQ ID NO: 56 - protein, 96 aa, Macropus rufus
FPDGEFIMQGCPECKLKENKYFSKLGAPIYQCMGCCFSRAYPTPARSKKTMLVPKNITSEATCCVAKAFTKATVMDNVKIENHTECHCSTCYYHKS

SEQ ID NO: 57 - protein, 118 aa, GENERIC
  misc_feature (1)..(1): F, V, or any other amino acid
XCXPTEYTXXXXXXEEAYCLTINTTICAGXCMTRDXNGKXXLXKXALSQDVCTYRXXXYXTXXIPGCPXHXXPYXSXPVAXSCKCGKCXTDYSDCXXEAXXXNYCTKPQXXXXXXFSV

SEQ ID NO: 58 - protein, 118 aa, Homo sapiens; identical to SEQ ID NO: 3

SEQ ID NO: 59 - protein, 118 aa, Bos taurus
FCIPTEYMMHVERKECAYCLTINTTVCAGYCMTRDVNGKLFLPKYALSQDVCTYRDFMYKTAEIPGCPRHVTPYFSYPVAISCKCGKCNTDYSDCIHEAIKTNYCTKPQKSYMVGFSI

SEQ ID NO: 60 - protein, 118 aa, Sus scrofa
FCIPTEYMMHVERKECAYCLTINTTICAGYCMTRDFNGKLFLPKYALSQDVCTYRDFMYKTVEIPGCPHHVTPYFSYPVAISCKCGKCNTDYSDCIHEAIKTNYCTKPQKSYVLEFSI

SEQ ID NO: 61 - protein, 118 aa, Llama glama
FCIPTEYMMHVERKECAYCLTINTTICAGYCMTRDFNGKLFLPKFALSQDVCTYRDFMYKTVEIPGCPHHVTPYFSYPVAVSCKCGKCDTDYSDCIQEAVKMNYCTKPQKPHVVGLSI

SEQ ID NO: 62 - protein, 118 aa, Canis familiaris
FCFPTEYTMHVERKECAYCLTINTTICAGYCMTRDINGKLFLPKYALSQDVCTYRDFMYKTVEIPGCPRHVTPYFSYPVAVSCKCGKCNTDYSDCIHEAIKTNYCTKPQKSYVVGFSI

SEQ ID NO: 63 - protein, 118 aa, Equus caballus
FCIPTEYMMHVERKECAYCLTINTTICAGYCMTRDINGKLFLPKYALSQDVCTYRDFMYKTVEIPGCPDHVTPYFSYPVAVSCKCGKCNTDYSDCIHEAIKANYCTKPQKSYVVEFSI

SEQ ID NO: 64 - protein, 118 aa, Rattus norvegicus
FCIPTEYMMYVDRRECAYCLTINTTICAGYCMTRDINGKLFLPKYALSQDVCTYRDFTYRTVEIPGCPHHVAPYFSYPVALSCKCGKCNTDYSDCTHEAVKTNYCTKPQTFYLGGFSG

SEQ ID NO: 65 - protein, 118 aa, Mus musculus
FCIPTEYTMYVDRRECAYCLTINTTICAGYCMTRDINGKLFLPKYALSQDVCTYRDFIYRTVEIPGCPHHVTPYFSFPVAVSCKCGKCNTDNSDCIHEAVRTNYCTKPQSFYLGGFSV

SEQ ID NO: 66 - protein, 114 aa, Gallus gallus
VCAPSEYTIHVEKRECAYCLAINTTICAGFCMTRDSNGKKLLLKSALSQNVCTYKEMFYQTALIPGCPHHTIPYYSYPVAISCKCGKCNTDYSDCVHEKVRTNYCTKPQKLCNM